(19) United States
(12) Patent Application Publication
(10) Pub. No.: US 2020/0163416 A1
(43) Pub. Date: May 28, 2020

(54) SHOELACE ADJUSTING DEVICE AND SHOE INCLUDING THE SAME

(71) Applicant: LG ELECTRONICS INC., Seoul (KR)

(72) Inventor: Eun KWON, Seoul (KR)

(73) Assignee: LG ELECTRONICS INC., Seoul (KR)

(21) Appl. No.: 16/632,832

(22) PCT Filed: Jul. 18, 2018
(86) PCT No.: PCT/KR2018/008138

§ 371 Date: Jan. 21, 2020

(30) Foreign Application Priority Data
Jul. 19, 2017 (KR) 10-2017-0091681

(51) Int. Cl. A43C 11/16; A61B 5/00; A43B 3/00; A61B 5/103

(52) U.S. Cl. CPC A43C 11/165; A61B 5/6807; A61B 2503/12; A61B 5/1038; A43B 3/0005

(57) ABSTRACT

The present disclosure relates to a shoelace adjusting device and a shoe including the same. The shoelace adjusting device and the shoe including the same based on an embodiment of the present disclosure include a motor configured to operate to adjust at least one part of the shoelace, a sensor including a motion sensor, and a processor configured to determine whether the shoe is being used in a running state, a walking state, or a sitting state based on sensing information from the motion sensor and to change a level for tightening the shoelace based on the respective states. Accordingly, the tightness of the shoelace may be automatically adjusted based on the state of the user wearing the shoes.

### TECHNICAL FIELD

The present disclosure relates to a shoelace adjusting device and a shoe including the same, and more particularly to a shoelace adjusting device capable of automatically adjusting the tightness of a shoelace based on the state of the user wearing the shoes and to a shoe including the same.

### BACKGROUND ART

Various kinds of wearable devices for providing convenience to users are being developed.

Meanwhile, shoes are products that users wear daily, and users wearing shoes perform various motions, such as walking, running, etc.

Meanwhile, a shoelace is used for a shoe in order to fasten a shoe, and the tightness of a shoelace is typically adjusted manually.

### DISCLOSURE

### Technical Problem

It is an object of the present disclosure to provide a shoelace adjusting device capable of automatically adjusting the tightness of a shoelace based on the state of the user wearing the shoes and a shoe including the same.

### Technical Solution

In order to accomplish the above object, a shoelace adjusting device and a shoe including the same based on an embodiment of the present disclosure include a motor configured to operate to adjust at least one part of the shoelace, a sensor including a motion sensor, and a processor configured to determine whether the shoe is being used in a running state, a walking state, or a sitting state based on sensing information from the motion sensor and to change a level for tightening the shoelace based on the respective states.

Meanwhile, in order to accomplish the above abject, a shoelace adjusting device and a shoe including the same based on another embodiment of the present disclosure include a first motor configured to operate to move at least one part of the shoelace, a second motor configured to rotate in the same direction as the first motor and to operate to move the at least one part of the shoelace, a sensor including a motion sensor, and a processor configured to determine whether the shoe is being used in a running state, a walking state, or a sitting state based on sensing information from the motion sensor and to change a level for tightening the shoelace based on the respective states.

Meanwhile, in order to accomplish the above abject, a shoelace adjusting device and a shoe including the same based on still another embodiment of the present disclosure include a shoelace adjuster configured to operate to adjust at least one part of the shoelace, a sensor including a motion sensor, and a processor configured to determine whether the shoe is being used in a running state, a walking state, or a sitting state based on sensing information from the motion sensor and to change a level for tightening the shoelace based on the respective states.

### Advantageous Effects

A shoelace adjusting device and a shoe including the same based on an embodiment of the present disclosure include a motor configured to operate to adjust at least one part of the shoelace, a sensor including a motion sensor, and a processor configured to determine whether the shoe is being used in a running state, a walking state, or a sitting state based on sensing information from the motion sensor and to change a level for tightening the shoelace based on the respective states, thereby automatically adjusting the tightness of the shoelace based on the state of the user wearing the shoes.

Meanwhile, the change in the level to which the shoelace is tightened is controlled based on flex-sensing information from a flex sensor, which detects the bent state of the shoelace, and state information of the shoe, thereby automatically adjusting the tightness of the shoelace so as to be suitable for the state of the user.

Meanwhile, whether the user is in a sitting state is further determined based on sensing information from the motion sensor and pressure sensing information from a pressure sensor, thereby automatically adjusting the tightness of the shoelace based on any of various states of the user wearing the shoes.

Meanwhile, in order to accomplish the above abject, a shoelace adjusting device and a shoe including the same based on another embodiment of the present disclosure include a first motor configured to operate to move at least one part of the shoelace, a second motor configured to rotate in the same direction as the first motor and to operate to move the at least one part of the shoelace, a sensor including a motion sensor, and a processor configured to determine whether the shoe is being used in a running state, a walking state, or a sitting state based on sensing information from the motion sensor and to change a level for tightening the shoelace based on the respective states, thereby automatically adjusting the tightness of the shoelace based on the state of the user wearing the shoes.

Meanwhile, in order to accomplish the above abject, a shoelace adjusting device and a shoe including the same based on still another embodiment of the present disclosure include a shoelace adjuster configured to operate to adjust at least one part of the shoelace, a sensor including a motion sensor, and a processor configured to determine whether the shoe is being used in a running state, a walking state, or a sitting state based on sensing information from the motion sensor and to change a level for tightening the shoelace based on the respective states, thereby automatically adjusting the tightness of the shoelace based on the state of the user wearing the shoes.

### DESCRIPTION OF DRAWINGS

FIG. 1 is a diagram showing a system that includes a shoelace adjusting device and a shoe including the same based on an embodiment of the present disclosure.

FIG. 2 is an enlarged view of the shoelace adjusting device and the shoe including the same in FIG. 1.

FIG. 3 is a schematic exemplary internal block diagram of the shoelace adjusting device in FIG. 2.

FIG. 4 is a view schematically illustrating the internal structure of the shoelace adjusting device in FIG. 2.

FIG. 5 is a flowchart illustrating a method of operating the shoelace adjusting device based on an embodiment of the present disclosure.

FIGS. 6 to 11B are views referenced to describe the method of operating the shoelace adjusting device in FIG. 5.

FIG. 12 is a schematic internal block diagram of another example of the shoelace adjusting device in FIG. 2.

FIGS. 13 to 16 are views referenced to describe the method of operating the shoelace adjusting device in FIG. 12.

FIG. 17 is a schematic internal block diagram of still another example of the shoelace adjusting device in FIG. 2.

FIG. 18 is a view referenced to describe the method of operating the shoelace adjusting device in FIG. 17.

### BEST MODE

Hereinafter, the present disclosure will be described in detail with reference to the accompanying drawings.

As used herein, the terms with which the names of components are suffixed, such as “module” and “unit”, are assigned to facilitate preparation of this specification, and are not intended to suggest unique meanings or functions. Accordingly, the terms “module” and “unit” may be used interchangeably.

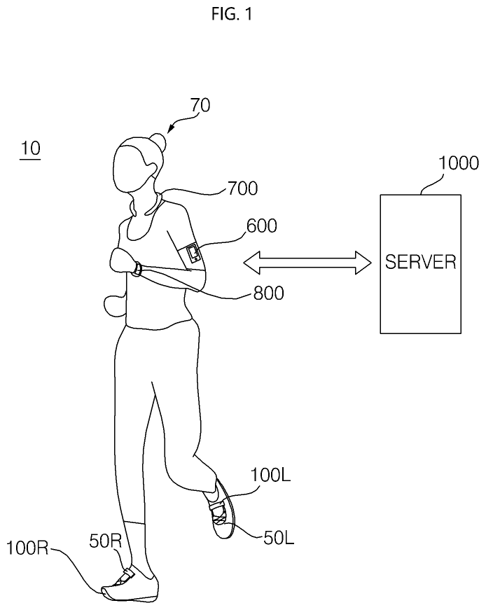

FIG. 1 is a diagram showing a system that includes a shoelace adjusting device and a shoe including the same based on an embodiment of the present disclosure.

Referring to the drawings, a system 10 in FIG. 1 is a shoelace-related system, and may include shoelace adjusting devices 100L and 100R disposed on respective shoes 50L and 50R of a user 70, a mobile terminal 600 configured to exchange data with the shoelace adjusting devices 100L and 100R, a smart watch 800, a wireless earphone 700, a server 1000, and the like.

The shoelace adjusting devices 100L and 100R are devices that may automatically adjust the shoelaces of the shoes 50L and 50R worn by the user 70 and may exchange data with at least one of the mobile terminal 600, the smart watch 800, or the wireless earphone 700, which is carried by the user.

Meanwhile, the server 1000 may exchange data with at least one of the mobile terminal 600, the smart watch 800, or the wireless earphone 700, which is carried by the user.

Meanwhile, each of the shoelace adjusting devices 100L and 100R based on the embodiment of the present disclosure may include a motor 155 configured to operate to adjust at least one part of the shoelace ASL, a sensor portion 130 including an acceleration sensor 134, and a processor 170 configured to determine whether the shoes 50 are being used in a running state, a walking state, or a sitting state based on sensing information from the acceleration sensor 134 and to change a level for tightening the shoelace ASL based on the respective states. Accordingly, the tightness of the shoelace ASL may be automatically adjusted based on the state of the user wearing the shoes 50.

Meanwhile, each of the shoelace adjusting devices 100L and 100R based on the embodiment of the present disclosure may change a level for tightening the shoelace ASL based on flex-sensing information from a flex sensor 132, which detects the bent state of the shoelace ASL, and state information of the shoes 50, thereby automatically adjusting the tightness of the shoelace ASL so as to be suitable for the state of the user.

Meanwhile, each of the shoelace adjusting devices 100L and 100R based on the embodiment of the present disclosure may further determine whether the user is in a sitting state based on sensing information from the acceleration sensor 134 and pressure sensing information from a pressure sensor 133, thereby automatically adjusting the tightness of the shoelace ASL based on any of various states of the user wearing the shoes 50.

Meanwhile, each of shoelace adjusting devices 100L and 100R based on another embodiment of the present disclosure may include a first motor 155 configured to operate to move at least one part of the shoelace ASL, a second motor 155 configured to rotate in the same direction as the first motor 155 and to operate to move the at least one part of the shoelace ASL, a sensor portion 130 including an acceleration sensor 134, and a processor 170 configured to determine whether the shoes 50 are being used in a running state, a walking state, or a sitting state based on sensing information from the acceleration sensor 134 and to change a level for tightening the shoelace ASL based on the respective states, thereby automatically adjusting the tightness of the shoelace ASL based on the state of the user wearing the shoes 50.

Meanwhile, each of shoelace adjusting devices 100L and 100R based on still another embodiment of the present disclosure may include a shoelace adjuster configured to operate to adjust at least one part of the shoelace ASL, a sensor portion 130 including an acceleration sensor 134, and a processor 170 configured to determine whether the shoes 50 are being used in a running state, a walking state, or a sitting state based on sensing information from the acceleration sensor 134 and to change a level for tightening the shoelace ASL based on the respective states, thereby automatically adjusting the tightness of the shoelace ASL based on the state of the user wearing the shoes 50.

Meanwhile, each of the shoelace adjusting devices 100L and 100R based on the embodiment of the present disclosure may calculate a stride speed, a gait angle, and a gait, and accordingly may provide various pieces of information.

Meanwhile, each of the shoelace adjusting devices 100L and 100R based on the embodiment of the present disclosure may transmit the behavior pattern of the user to the mobile terminal 600, may measure the amount of exercise, or may measure a gait.

Meanwhile, each of the shoelace adjusting devices 100L and 100R based on the embodiment of the present disclosure may automatically adjust the tightness of the shoelace based on a shoelace tightening level set through the mobile terminal 600.

Meanwhile, each of the shoelace adjusting devices 100L and 100R based on the embodiment of the present disclosure may adjust the shoelace tightening level and may store the current shoelace tightening level in the mobile terminal 600.

Meanwhile, each of the shoelace adjusting devices 100L and 100R based on the embodiment of the present disclosure may identify the tightening level based on the type of shoe, and may change the tightening level based on the type of shoe.

Meanwhile, each of the shoelace adjusting devices 100L and 100R based on the embodiment of the present disclosure may identify the shoe wearer, and may change the tightening level based on the type of shoe and on the wearer.

Meanwhile, each of the shoelace adjusting devices 100L and 100R based on the embodiment of the present disclosure may generate vibration in order to prevent separation of the mobile terminal 600, particularly, prevent loss thereof when the distance to the mobile terminal 600 is equal to or greater than a predetermined value, i.e. when the intensity of wireless signals exchanged with the paired mobile terminal 600 is equal to or less than a reference value.

Meanwhile, based on the embodiment of the present disclosure, in the state in which the shoes are taken off, the mobile terminal 600 may generate vibration in order to prevent separation of the shoelace adjusting devices 100L and 100R, particularly, prevent loss thereof when the distance to the shoelace adjusting devices 100L and 100R is equal to or greater than a predetermined value, i.e. when the intensity of wireless signals exchanged with the paired shoelace adjusting devices 100L and 100R is equal to or less than a reference value.

Meanwhile, in the state in which the wearer takes off the shoes, each of the shoelace adjusting devices 100L and 100R based on the embodiment of the present disclosure may tighten the shoelace to the maximum extent in order to prevent other persons from putting on the shoes.

Meanwhile, each of the shoelace adjusting devices 100L and 100R based on the embodiment of the present disclosure may automatically adjust the tightness of the shoelace based on position information, such as GPS information, received from the mobile terminal 600 or the like.

In one example, when the current position is an intersection, each of the shoelace adjusting devices 100L and 100R may perform control such that the shoelace is tightened.

In another example, when the current position is near the home, each of the shoelace adjusting devices 100L and 100R may perform control such that the shoelace is loosened.

Meanwhile, each of the shoelace adjusting devices 100L and 100R based on the embodiment of the present disclosure may automatically adjust the tightness of the shoelace based on the surrounding environment.

In one example, when an obstacle is present ahead, each of the shoelace adjusting devices 100L and 100R may perform control such that the shoelace is tightened.

In another example, when it is time for the user to get out of a subway, a bus, a car, or the like, each of the shoelace adjusting devices 100L and 100R may tighten the shoelace, which is in a loosened state, so that the user recognizes that it is time to get out.

Meanwhile, each of the shoelace adjusting devices 100L and 100R based on the embodiment of the present disclosure may automatically adjust the tightness of the shoelace in order to correct a gait or the like.

For example, upon detecting an out-toed gait, an in-toed gait, or the like, each of the shoelace adjusting devices 100L and 100R may automatically adjust the tightness of the shoelace in order to correct a gait or the like. Specifically, the tightness of the shoelace may be automatically adjusted in the manner of asymmetrically tightening one part and the other part of the shoelace.

Meanwhile, each of the shoelace adjusting devices 100L and 100R based on the embodiment of the present disclosure may automatically adjust the tightness of the shoelace based on the exercise state of the wearer. Accordingly, it may be helpful for managing the pace of the user.

Meanwhile, the mobile terminal 600 based on the embodiment of the present disclosure may receive data from the shoelace adjusting devices 100L and 100R and may store the data. In particular, the shoelace adjustment levels and the like may be stored.

Meanwhile, the mobile terminal 600 based on the embodiment of the present disclosure may identify the behavior pattern of the user, may measure the amount of exercise of the user, or may measure the gait of the user using data received from the shoelace adjusting devices 100L and 100R.

In addition, the mobile terminal 600 based on the embodiment of the present disclosure may provide the current position information, the generated map, the stride speed, the gait angle, and the gait using data received from the shoelace adjusting devices 100L and 100R, and accordingly, various pieces of information may be provided. Thus, the convenience of the user may be enhanced.

Meanwhile, the mobile terminal 600 based on the embodiment of the present disclosure may transmit information about the set shoelace tightening levels to the shoelace adjusting devices 100L and 100R.

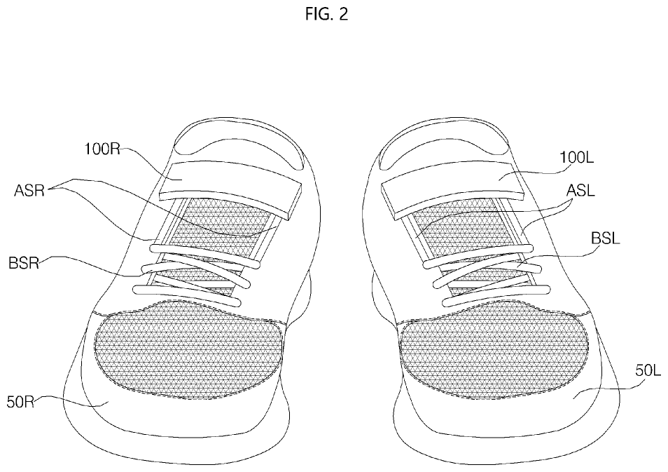

FIG. 2 is an enlarged view of the shoelace adjusting device and the shoe including the same in FIG. 1.

Referring to the drawing, a left shoelace ASL and a right shoelace ASR are attached to the left shoe 50L and the right shoe 50R, respectively.

Meanwhile, based on the embodiment of the present disclosure, a left shoelace adjusting device 100L and a right shoelace adjusting device 100R are respectively provided to automatically adjust the left shoelace ASL and the right shoelace ASR.

The left shoelace adjusting device 100L and the right shoelace adjusting device 100R may be, respectively, in contact with one part of the left shoelace ASL and one part of the right shoelace ASR.

Meanwhile, in the drawing, the shoelaces are exemplarily illustrated as being divided into fixed-type shoelaces BSL and BSR, which are crossed and are difficult to adjust, and adjustable shoelaces ASL and ASR.

The left shoelace adjusting device 100L and the right shoelace adjusting device 100R are provided with the adjustable shoelaces ASL and ASR.

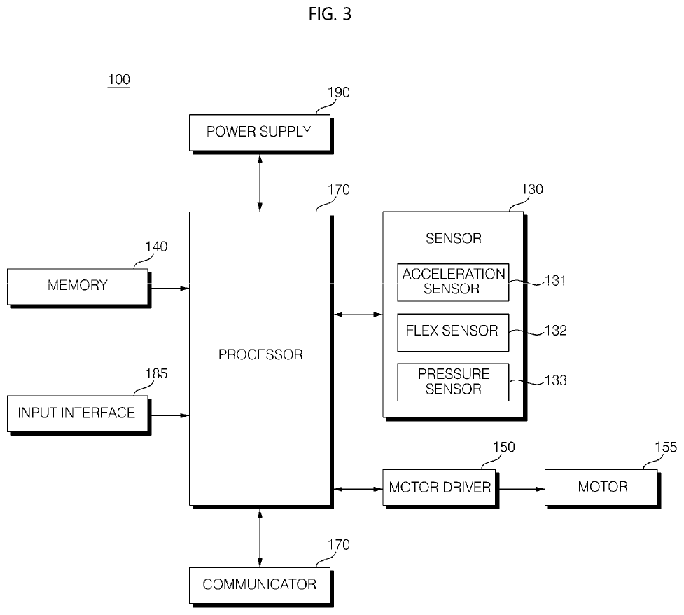

Hereinafter, any one of the left shoelace adjusting device 100L and the right shoelace adjusting device 100R will be described with reference to FIG. 3.

FIG. 3 is a schematic exemplary internal block diagram of the shoelace adjusting device in FIG. 2.

Referring to the drawing, the shoelace adjusting device 100 may include a sensor portion 130, a communicator 135, a memory 140, a motor driver 150, a motor 155, a processor 170, an input interface 185, and a power supply 190. When these components are implemented in practice, two or more components may be combined into a single component, or one component may be subdivided into two or more components, if necessary.

The sensor portion 130 may include a motion sensor 131, a flex sensor 132, a pressure sensor 133, and the like.

The motion sensor 131 may include an acceleration sensor, a gyro sensor, a gravity sensor, and the like. In particular, the motion sensor 131 may include a six-axis sensor.

The motion sensor 131 may output motion information of the shoelace adjusting device 100, e.g. motion information (acceleration information or angular velocity information) or position information on the basis of x-, y- and z-axes.

The flex sensor 132 may detect the bent state of the shoelace ASL.

The pressure sensor 133 may detect pressure applied to the shoelace adjusting device 100. For example, the pressure sensor 133 may detect pressure generated by the top of the foot of the shoe wearer.

Meanwhile, the communicator 135 may exchange data with an external electronic device.

In particular, the communicator 135 may exchange data with the mobile terminal 600. To this end, the communicator 135 may undergo pairing with the mobile terminal 600.

Meanwhile, the communicator 135 may provide an interface for communication with an external device. To this end, the communicator 135 may include at least one of a mobile communication module (not shown), a wireless Internet module (not shown), a short-range communication module (not shown), or a GPS module (not shown).

For example, the communicator 135 may perform Bluetooth communication, Wi-Fi communication, low-power wide-area communication, and the like, and may thus transmit information sensed by the shoelace adjusting device 100 to the paired mobile terminal 600.

The memory 140 may store a program for the processing or control of the processor 170 of the shoelace adjusting device 100, and may also function to temporarily store input/output data.

In addition, the memory 140 may store shoelace adjustment level information, stride information, stride speed information, gait angle information, gait information, and the like.

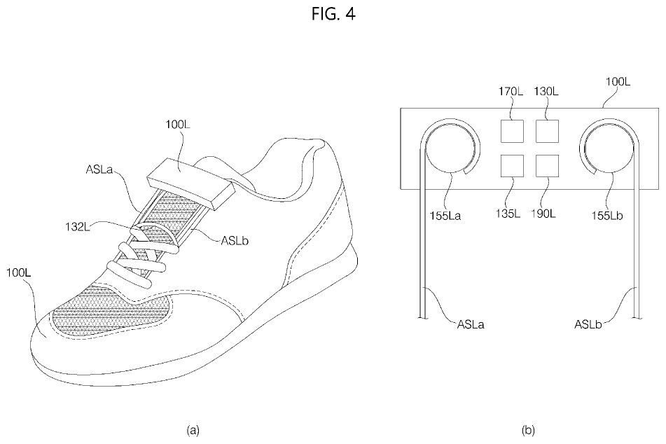

The motor driver 150 drives the motor 155 such that the motor 155 rotates. For example, as shown in FIG. 4, when two motors 155La and 155Lb are provided, the motor driver 150 may perform control such that the two motors rotate in opposite directions.

The processor 170 may control the operation of each unit in the shoelace adjusting device 100 so as to control the overall operation of the shoelace adjusting device 100.

Meanwhile, the processor 170 may determine whether the shoes 50 are being used in a running state, a walking state, or a sitting state based on sensing information from the motion sensor 131, and may change a level for tightening the shoelace ASL based on the respective states.

Meanwhile, the processor 170 may change a level for tightening the shoelace ASL based on flex-sensing information from the flex sensor 132 and state information of the shoe 50.

Meanwhile, the processor 170 may determine whether the shoes 50 are being used in a running state, a walking state, or a sitting state based on sensing information from the motion sensor 131 and pressure sensing information from the pressure sensor 133, and may change a level for tightening the shoelace ASL based on the respective states.

Meanwhile, when the pressure sensing information is equal to or greater than a first reference value for a predetermined period of time, the processor 170 may determine that the feet of the wearer of the shoes 50 are in a swollen state, and may change a level for tightening the shoelace ASL in response to the swollen state.

Meanwhile, the processor 170 may further determine whether the user is taking off the shoes 50 or putting on the shoes 50 based on sensing information from the motion sensor 131, and may change a level for tightening the shoelace ASL based on the taking-off state or the putting-on state.

Meanwhile, the processor 170 may compare state information of the wearer of the shoes 50, received from the mobile terminal 60, with state information, determined based on sensing information from the motion sensor 131, may calculate final state information, and may change a level for tightening the shoelace ASL based on the final state information.

Meanwhile, the processor 170 may receive temperature information from the mobile terminal 600, and may change the level to which the shoelace ASL is tightened based on the received temperature information.

Meanwhile, the processor 170 may receive temperature information from the mobile terminal 600, and may change a level for tightening the shoelace ASL based on the received temperature information.

Meanwhile, the processor 170 may receive humidity information from the mobile terminal 600, and may change a level for tightening the shoelace ASL based on the received humidity information.

Meanwhile, when the motor 155 includes the first motor and the second motor, the processor 170 may perform control such that the left side of the shoelace ASL and the right side of the shoelace ASL are tightened differently based on sensing information from the motion sensor 131.

Meanwhile, the processor 170 may change a level for tightening the shoelace ASL based on the moving speed of the shoe 50.

Meanwhile, the processor 170 may determine whether the shoelace ASL is the shoelace ASL of the left shoe 50 or the shoelace ASL of the right shoe 50 based on sensing information from the motion sensor 131, and may change a level for tightening the shoelace ASL based on the determined information.

Meanwhile, the processor 170 may receive tightness information of the shoelace ASL from the mobile terminal 600, and may change a level for tightening the shoelace ASL based on the received tightness information of the shoelace ASL.

Meanwhile, the processor 170 may calculate a stride speed, a gait angle, and a gait, and accordingly may provide various pieces of information.

Meanwhile, the processor 170 may transmit the behavior pattern of the user to the mobile terminal 600, may measure the amount of exercise, or may measure a gait.

Meanwhile, the processor 170 may automatically adjust the tightness of the shoelace based on a shoelace tightening level set through the mobile terminal 600.

Meanwhile, the processor 170 may identify the tightening level based on the type of shoe, and may change the tightening level based on the type of shoe.

Meanwhile, the processor 170 may identify the shoe wearer, and may change the tightening level based on the type of shoe and on the wearer.

Meanwhile, the processor 170 may generate vibration in order to prevent separation of the mobile terminal 600, particularly, prevent loss thereof when the distance to the mobile terminal 600 is equal to or greater than a predetermined value, i.e. when the intensity of wireless signals exchanged with the paired mobile terminal 600 is equal to or less than a reference value.

Meanwhile, in the state in which the wearer takes off the shoes, the processor 170 may tighten the shoelace to the maximum extent in order to prevent other persons from putting on the shoes.

Meanwhile, the processor 170 may automatically adjust the tightness of the shoelace based on position information, such as GPS information, received from the mobile terminal 600 or the like.

In one example, when the current position is an intersection, each shoelace adjusting device 100 may perform control such that the shoelace is tightened.

In another example, when the current position is near the home, each shoelace adjusting device 100 may perform control such that the shoelace is loosened.

Meanwhile, the processor 170 may automatically adjust the tightness of the shoelace based on the surrounding environment.

In one example, when an obstacle is present ahead, each shoelace adjusting device 100 may perform control such that the shoelace is tightened.

In another example, when it is time for the user to get out of a subway, a bus, a car, or the like, each shoelace adjusting device 100 may tighten the shoelace, which is in a loosened state, so that the user recognizes that it is time to get out.

Meanwhile, the processor 170 may automatically adjust the tightness of the shoelace in order to correct a gait or the like.

For example, upon detecting an out-toed gait, an in-toed gait, or the like, each shoelace adjusting device 100 may automatically adjust the tightness of the shoelace in order to correct a gait or the like. Specifically, the tightness of the shoelace may be automatically adjusted in the manner of asymmetrically tightening one part and the other part of the shoelace.

Meanwhile, the processor 170 may automatically adjust the tightness of the shoelace based on the exercise state of the wearer. Accordingly, it may be helpful for managing the pace of the user.

Meanwhile, the input interface 185 may include a button for initializing the shoelace adjusting device 100 or inputting an operation.

The power supply 190 may supply power required for operation of the respective components under the control of the processor 170.

Meanwhile, the power supply 190 may include a battery for storing and outputting DC power.

FIG. 4 is a view schematically illustrating the internal structure of the shoelace adjusting device in FIG. 2.

Referring to the drawing, the left shoe 50L may be provided with shoelaces ASLa and ASLb, and the left shoelace adjusting device 100L may be disposed at part portions of the shoelaces ASLa and ASLb.

Meanwhile, the flex sensor 132L for detecting bending may be disposed at the other part portions of the shoelaces ASLa and ASLb.

The left shoelace adjusting device 100L may include therein a processor 170L, a sensor portion 130L, a communicator 135L, a power supply 190L, and motors 155La and 155Lb configured to rotate in opposite directions.

Meanwhile, one part portion of each of the shoelaces ASLa and ASLb is connected to a corresponding one of the motors 155La and 155Lb, and the shoelaces ASLa and ASLb are loosened or tightened based on rotation of the motors 155La and 155Lb.

For example, the shoelaces ASLa and ASLb are tightened by rightward rotation of the motor 155La and leftward rotation of the motor 155Lb, and are loosened by leftward rotation of the motor 155La and rightward rotation of the motor 155Lb.

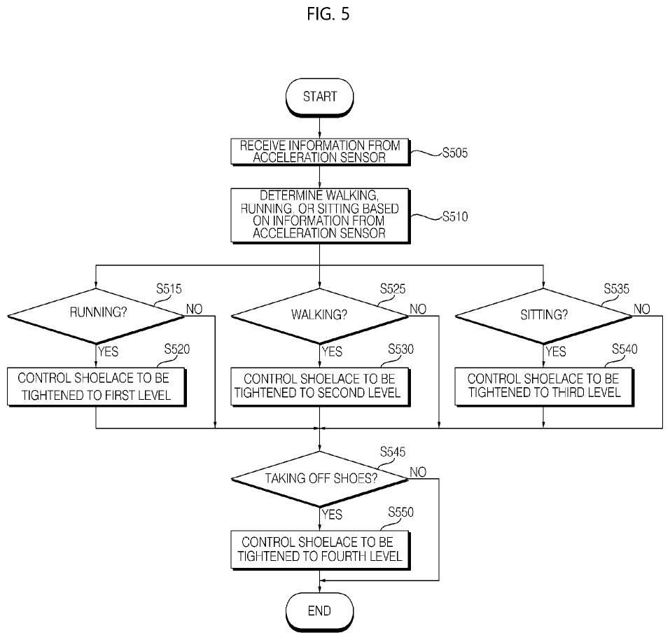

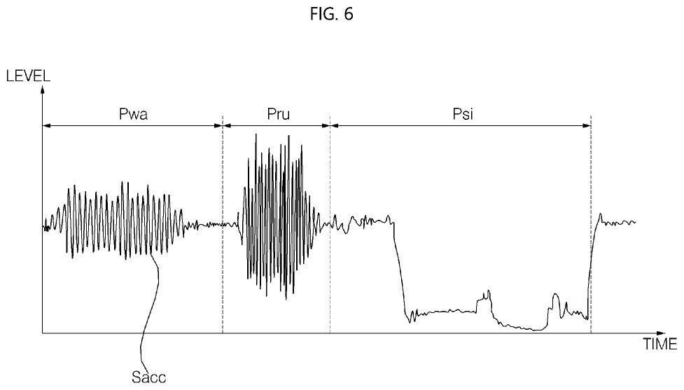

FIG. 5 is a flowchart illustrating a method of operating the shoelace adjusting device based on an embodiment of the present disclosure, and FIGS. 6 to 11B are views referenced to describe the method of operating the shoelace adjusting device in FIG. 5.

Referring to the drawing, the processor 170 receives sensing information from the motion sensor 131 (S505). In detail, motion information may be received.

The motion information may conceptually include acceleration information from the acceleration sensor, angular velocity information from the gyro sensor, and the like.

Subsequently, the processor 170 determines whether the shoes 50 are being used in a running state, a walking state, or a sitting state based on sensing information from the motion sensor 131 (S510).

For example, in the motion information Sacc shown in FIG. 6, the motion information corresponding to the section PWa indicates a walking state, the motion information corresponding to the section Pru indicates a running state, and the motion information corresponding to the section Psi indicates a sitting state.

Accordingly, the processor 170 may determine the section in FIG. 6 to which the sensing information from the motion sensor 131 corresponds, and may determine whether the shoes 50 are being used in a running state, a walking state, or a sitting state.

Subsequently, when the shoes 50 are being used in the running state (S515), the processor 170 may perform control such that the shoelaces are tightened to a first level (S520).

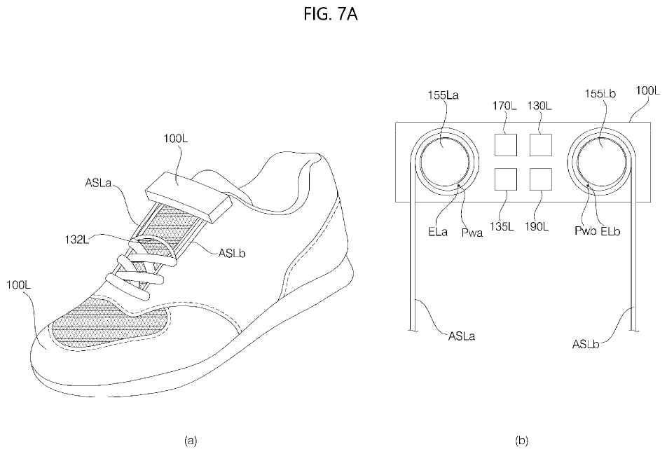

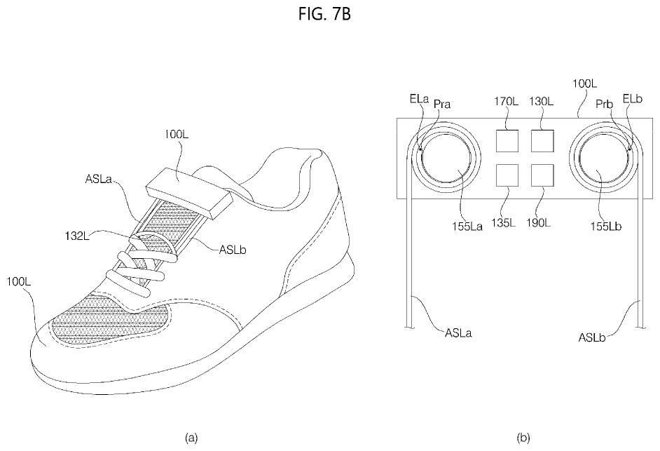

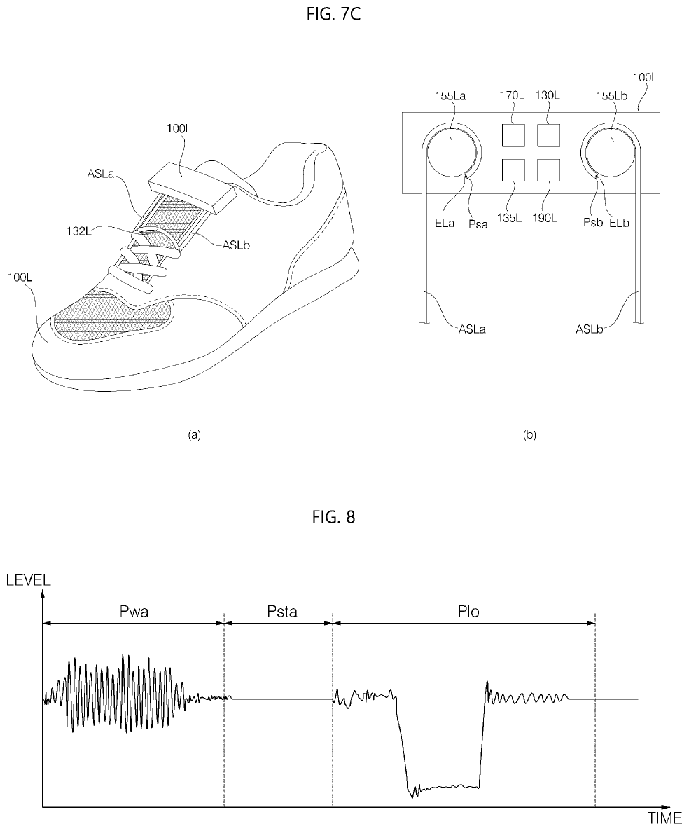

For example, as shown in FIG. 7B, the processor 170 may perform control such that the motor 155La rotates to the right and the motor 155Lb rotates to the left. In particular, the processor 170 may perform control such that the part portions ELA and ELb of the shoelaces ASLa and ASLb are, respectively, wound to the positions Pra and Prb. Accordingly, the shoelaces are tightly wound automatically.

Subsequently, when the shoes 50 are being used in the walking state (S525), the processor 170 may perform control such that the shoelaces are tightened to a second level (S530).

For example, as shown in FIG. 7A, the processor 170 may perform control such that the motor 155La rotates to the right and the motor 155Lb rotates to the left. In particular, the processor 170 may perform control such that the part portions ELA and ELb of the shoelaces ASLa and ASLb are, respectively, wound to the positions Pwa and Pwb. Accordingly, the shoelaces are slightly tightly wound automatically.

Subsequently, when the user is in the sitting state (S535), the processor 170 may perform control such that the shoelaces are tightened to a third level (S540).

For example, as shown in FIG. 7C, the processor 170 may perform control such that the motor 155La rotates to the right and the motor 155Lb rotates to the left. In particular, the processor 170 may perform control such that the part portions ELA and ELb of the shoelaces ASLa and ASLb are, respectively, wound to the positions Psa and Psb. Accordingly, the shoelaces are automatically loosened.

Comparing FIGS. 7A to 7C, the shoelaces are loosened so as to have the longest length in the sitting state shown in FIG. 7C, the shoelaces are wound so as to have an intermediate length in the walking state shown in FIG. 7A, and the shoelaces are most tightly wound so as to have the shortest length in the running state shown in FIG. 7A. As such, the shoelace tightening level is automatically changed, thereby enhancing the convenience of the user.

Meanwhile, the processor 170 may change the level for tightening the shoelaces ASL based on flex-sensing information from the flex sensor 132 and state information of the shoe 50. In particular, fine tightening may be performed.

For example, the processor 170 may determine that when the level of the flex-sensing information from the flex sensor 132 increases, the shoelaces are further loosened, and when the level of the flex-sensing information decreases, the shoelaces are further tightened.

Meanwhile, whether the user is in a sitting state may be determined based on sensing information from the motion sensor 131, such as the section Psi in FIG. 6, and in addition, whether the shoes 50 are being used in a running state, a walking state, or a sitting state may be determined based on pressure sensing information from the pressure sensor 133.

For example, in the running state, the pressure generated by the top of the foot is large, and in the sitting state, the pressure generated by the top of the foot is small. Using this, the processor 170 may distinguish among the running state, the walking state, and the sitting state.

In addition, the processor 170 may determine whether the shoes 50 are being used in the running state, the walking state, or the sitting state based on sensing information from the motion sensor 131 and pressure sensing information from the pressure sensor 133, and may control a change in the level for tightening the shoelaces ASL based on the respective states. Accordingly, it is possible to more accurately identify the respective states and to change the level for tightening the shoelaces ASL so as to be suitable for the respective states.

Meanwhile, when the pressure sensing information is equal to or greater than the first reference value for a predetermined period of time, the processor 170 may determine that the feet of the wearer of the shoes 50 are in a swollen state, and may control a change in the level for tightening the shoelaces ASL in response to the swollen state.

Meanwhile, when the user takes off the shoes (S545), the processor 170 may perform control such that the shoelaces are tightened to a fourth level, at which the shoelaces are in the maximally loosened state (S550).

In one example, the processor 170 may further determine whether the user is taking off the shoes 50 or putting on the shoes 50 based on sensing information from the motion sensor 131, and may control a change in the level for tightening the shoelaces ASL based on the taking-off state or the putting-on state.

In another example, the processor 170 may further determine whether the user is taking off the shoes 50 or putting on the shoes 50 based on sensing information from the motion sensor 131 and pressure sensing information from the pressure sensor 133, and may control a change in the level for tightening the shoelaces ASL based on the taking-off state or the putting-on state.

FIG. 8 illustrates motion information when the user walks and takes off the shoes.

Referring to the drawing, the motion information corresponding to the section PWa indicates a walking state, the motion information corresponding to the section Psta indicates a stationary state, and the motion information corresponding to the section Plo indicates a state of taking off the shoes.

Accordingly, the processor 170 may determine the section in FIG. 8 to which the sensing information from the motion sensor 131 corresponds, and may determine whether the shoes 50 are being used in a walking state or a stationary state or are being taken off.

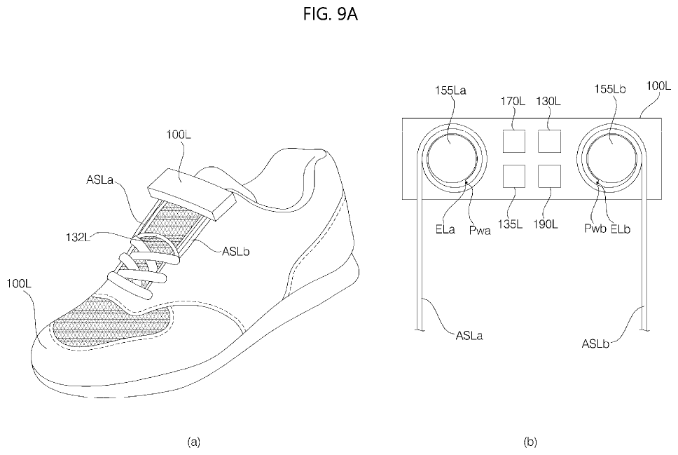

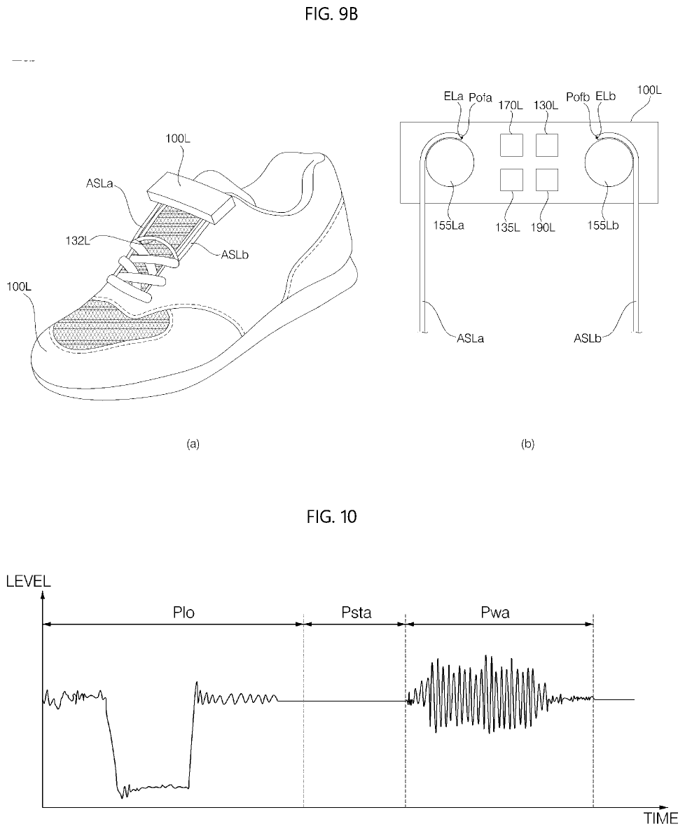

For example, as indicated by the section PWa, in the walking state, the processor 170, as shown in FIG. 9A, may perform control such that the motor 155La rotates to the right and the motor 155Lb rotates to the left. In particular, the processor 170 may perform control such that the part portions ELA and ELb of the shoelaces ASLa and ASLb are, respectively, wound to the positions Pwa and Pwb. Accordingly, the shoelaces are slightly tightly wound automatically.

Meanwhile, as indicated by the section Plo, in the state of taking off the shoes, the processor 170, as shown in FIG. 9B, may perform control such that the motor 155La rotates to the right and the motor 155Lb rotates to the left. In particular, the processor 170 may perform control such that the part portions ELA and ELb of the shoelaces ASLa and ASLb are, respectively, wound to the positions Pofa and Pofb. Accordingly, the shoelaces are automatically loosened to the maximum extent.

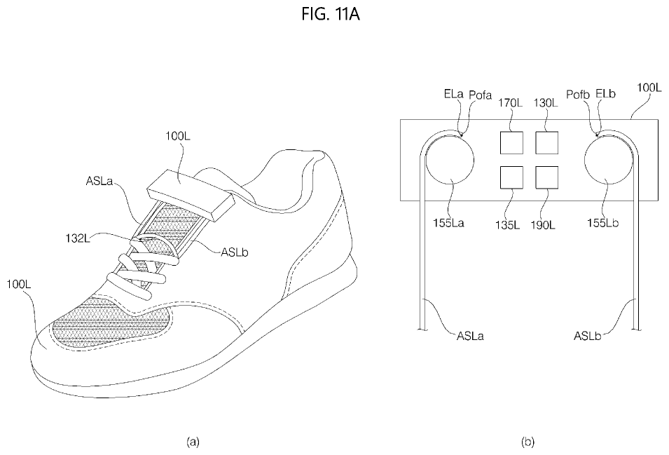

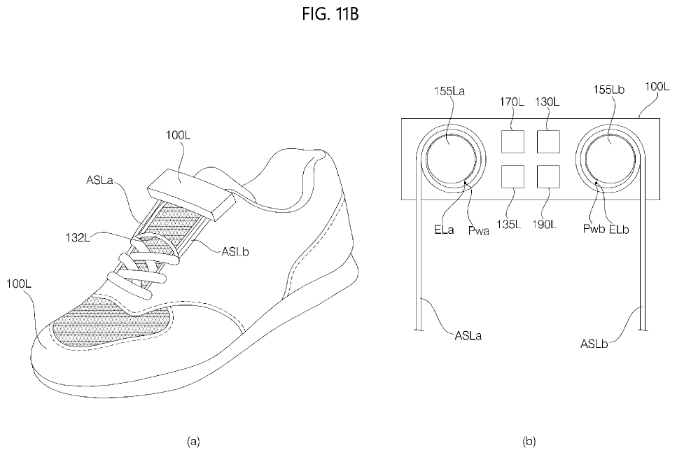

FIG. 10 illustrates motion information when the user puts on the shoes and walks.

Referring to the drawing, the motion information corresponding to the section Plo indicates a state of putting on the shoes, the motion information corresponding to the section Psta indicates a stationary state, and the motion information corresponding to the section PWa indicates a walking state.

Accordingly, the processor 170 may determine the section in FIG. 10 to which the sensing information from the motion sensor 131 corresponds, and may determine whether the shoes 50 are being put on or are being used in a stationary state or a walking state.

For example, as indicated by the section Plo, in the state of putting on the shoes, the processor 170, as shown in FIG. 11A, may perform control such that the motor 155La rotates to the right and the motor 155Lb rotates to the left. In particular, the processor 170 may perform control such that the part portions ELA and ELb of the shoelaces ASLa and ASLb are, respectively, located to the positions Pofa and Pofb. Accordingly, the shoelaces are automatically loosened to the maximum extent.

Subsequently, as indicated by the section PWa, in the walking state, the processor 170, as shown in FIG. 9B, may perform control such that the motor 155La rotates to the right and the motor 155Lb rotates to the left. In particular, the processor 170 may perform control such that the part portions ELA and ELb of the shoelaces ASLa and ASLb are, respectively, wound to the positions Pwa and Pwb. Accordingly, the shoelaces are slightly tightly wound automatically.

Meanwhile, the processor 170 may control a change in the level for tightening the shoelaces ASL based on the moving speed of the shoe 50.

Meanwhile, the processor 170 may determine whether the shoelaces ASL are the shoelaces ASL of the left shoe 50 or the shoelaces ASL of the right shoe 50 based on sensing information from the motion sensor 131, and may control a change in the level for tightening the shoelaces ASL based on the determined information.

Meanwhile, the processor 170 may identify the tightening level based on the type of shoe, and may change the tightening level based on the type of shoe.

Meanwhile, the processor 170 may identify the shoe wearer, and may change the tightening level based on the type of shoe and on the wearer.

Meanwhile, in the state in which the wearer takes off the shoes, the processor 170 may tighten the shoelaces to the maximum extent in order to prevent other persons from putting on the shoes.

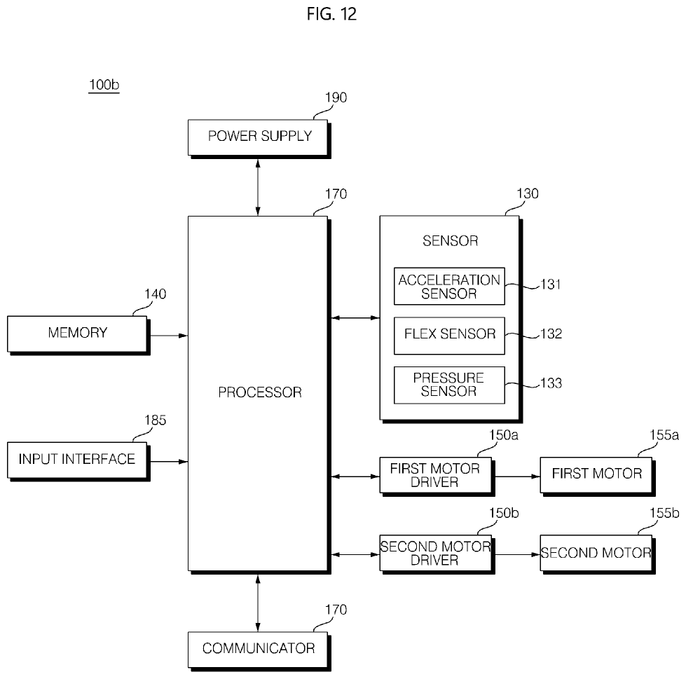

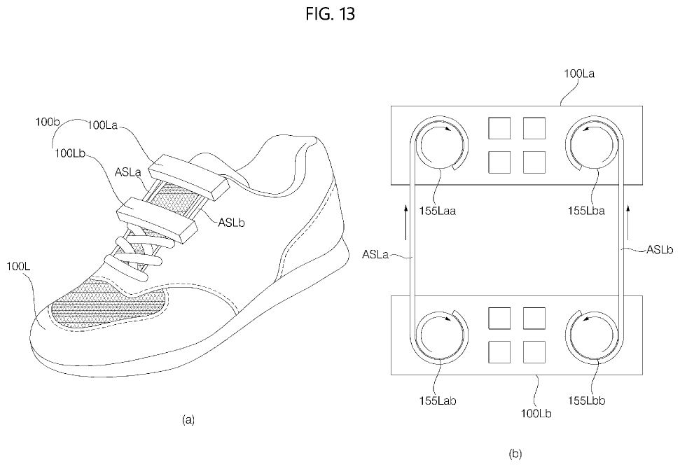

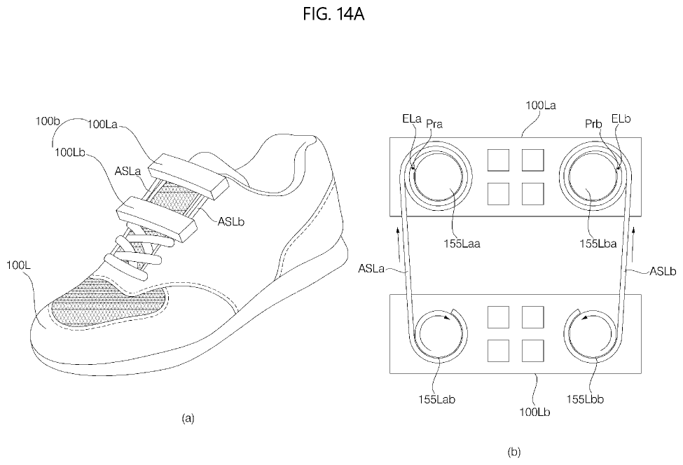

FIG. 12 is a schematic internal block diagram of another example of the shoelace adjusting device in FIG. 2, and FIGS. 13 to 16 are views referenced to describe the method of operating the shoelace adjusting device in FIG. 12.

Referring to FIG. 12, the shoelace adjusting device 100b in FIG. 12 is similar to the shoelace adjusting device 100 in FIG. 3, but is different in that a first motor driver 150a for driving the first motor 155a and a second motor driver 150b for driving the second motor 155b are further provided.

Here, the second motor 155b is characterized in that it is rotated in the same manner as the first motor 155a.

That is, as shown in FIG. 13, the shoelace adjusting device 100b may include a first portion 100La, in which the first motor 155a is disposed, and a second portion 100Lb, in which the second motor 155b is disposed. The first portion 100La and the second portion 100Lb may be spaced apart from each other.

Unlike FIG. 4, referring to FIG. 13, in order to adjust the shoelaces ALSla and ASLb, the first motor 155a and the second motor 155b, which are disposed at opposite ends of the shoelaces ALSla and ASLb, are rotated in the same direction. Accordingly, the shoelace tightening level may be more rapidly changed.

Meanwhile, the shoelace adjusting device 100b may determine whether the shoes 50 are being used in a running state, a walking state, or a sitting state based on sensing information from the motion sensor 131, and may control a change in the level for tightening the shoelaces ASL based on the respective states. Accordingly, the tightness of the shoelaces ASL may be automatically adjusted based on the state of the user wearing the shoes 50.

For example, when the shoes 50 are being used in the running state, the processor 170 may perform control such that the shoelaces are tightened to the first level.

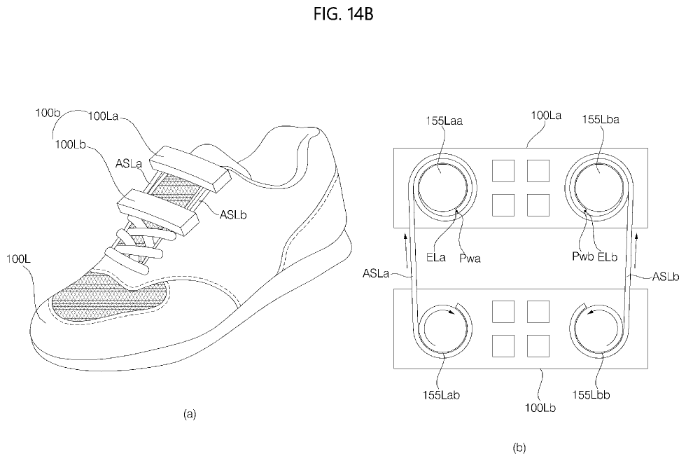

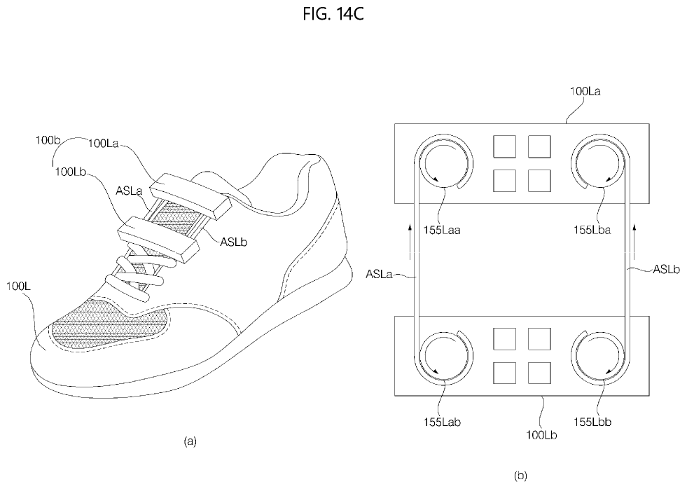

For example, as shown in FIG. 14B, the processor 170 may perform control such that the motors 155Laa and 155Lab rotate to the right and the motors 155Lba and 155Lbb rotate to the left. In particular, the processor 170 may perform control such that the part portions ELA and ELb of the shoelaces ASLa and ASLb are, respectively, wound to the positions Pra and Prb. Accordingly, the shoelaces are tightly wound automatically.

Subsequently, when the shoes 50 are being used in the walking state, the processor 170 may perform control such that the shoelaces are tightened to the second level.

For example, as shown in FIG. 14A, the processor 170 may perform control such that the motors 155Laa and 155Lab rotate to the right and the motors 155Lba and 155Lbb rotate to the left. In particular, the processor 170 may perform control such that the part portions ELA and ELb of the shoelaces ASLa and ASLb are, respectively, wound to the positions Pwa and Pwb. Accordingly, the shoelaces are slightly tightly wound automatically.

Subsequently, when the user is in the sitting state, the processor 170 may perform control such that the shoelaces are tightened to the third level.

For example, as shown in FIG. 14C, the processor 170 may perform control such that the motors 155Laa and 155Lab rotate to the right and the motors 155Lba and 155Lbb rotate to the left. In particular, the processor 170 may perform control such that the part portions ELA and ELb of the shoelaces ASLa and ASLb are, respectively, wound to the positions Psa and Psb. Accordingly, the shoelaces are automatically loosened.

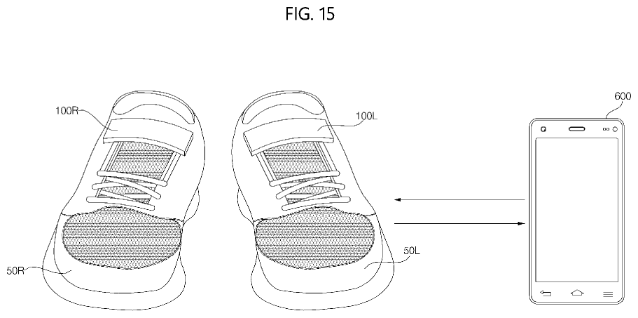

Meanwhile, as shown in FIG. 15, the left shoelace adjusting device 100L and the right shoelace adjusting device 100R may exchange data with the mobile terminal 600.

Meanwhile, the processor 170 may compare state information of the wearer of the shoes 50, received from the mobile terminal 60, with state information, determined based on sensing information from the motion sensor 131, may calculate final state information, and may control a change in the level for tightening the shoelaces ASL based on the final state information.

Meanwhile, the processor 170 may receive temperature information from the mobile terminal 600, and may change the level for tightening the shoelaces ASL based on the received temperature information.

Meanwhile, the processor 170 may receive temperature information from the mobile terminal 600, and may control a change in the level for tightening the shoelaces ASL based on the received temperature information.

Meanwhile, the processor 170 may receive humidity information from the mobile terminal 600, and may control a change in the level for tightening the shoelaces ASL based on the received humidity information.

Meanwhile, the processor 170 may receive tightness information of the shoelaces ASL from the mobile terminal 600, and may control a change in the level for tightening the shoelaces ASL based on the received tightness information of the shoelaces ASL.

Meanwhile, the processor 170 may calculate a stride speed, a gait angle, and a gait, and accordingly may provide various pieces of information.

Meanwhile, the processor 170 may transmit the behavior pattern of the user to the .mobile terminal 600, may measure the amount of exercise, or may measure a gait.

Meanwhile, the processor 170 may automatically adjust the tightness of the shoelaces based on a shoelace tightening level set through the mobile terminal 600.

Meanwhile, the processor 170 may generate vibration in order to prevent separation of the mobile terminal 600, particularly, prevent loss thereof when the distance to the mobile terminal 600 is equal to or greater than a predetermined value, i.e. when the intensity of wireless signals exchanged with the paired mobile terminal 600 is equal to or less than a reference value.

Meanwhile, the processor 170 may automatically adjust the tightness of the shoelaces based on position information, such as GPS information, received from the mobile terminal 600 or the like.

In one example, when the current position is an intersection, each shoelace adjusting device 100 may perform control such that the shoelaces are tightened.

In another example, when the current position is near the home, each shoelace adjusting device 100 may perform control such that the shoelaces are loosened.

Meanwhile, the processor 170 may automatically adjust the tightness of the shoelaces based on the surrounding environment.

In one example, when an obstacle is present ahead, each shoelace adjusting device 100 may perform control such that the shoelaces are tightened.

In another example, when it is time for the user to get out of a subway, a bus, a car, or the like, each shoelace adjusting device 100 may tighten the shoelaces, which are in a loosened state, so that the user recognizes that it is time to get out.

Meanwhile, the processor 170 may automatically adjust the tightness of the shoelaces based on the exercise state of the wearer. Accordingly, it may be helpful for managing the pace of the user.

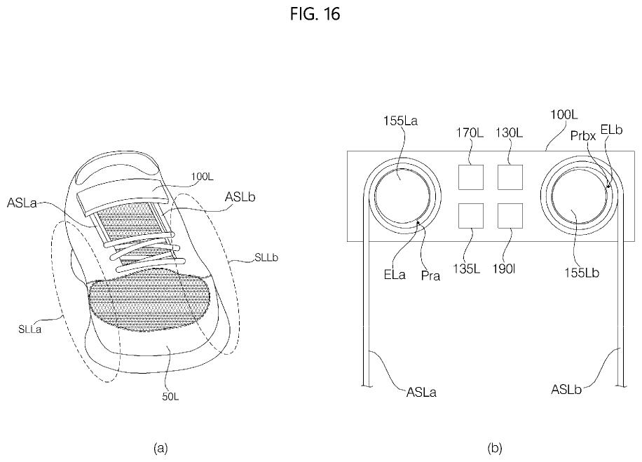

FIG. 16 illustrates that the left side and the right side of the shoelaces ALSa and ASLb of the shoe 50L are tightened differently.

For example, similar to FIG. 12, when the motor 155 includes the first motor 155a and the second motor 155b, the processor 170 may perform control such that the left side and the right side of the shoelaces ALSa and ASLb are tightened differently based on sensing information from the motion sensor 131.

For example, when the user has an asymmetrical gait, the processor 170 may automatically adjust the tightness of the shoelaces in order to correct the gait or the like.

For example, upon detecting an out-toed gait, an in-toed gait, or the like, the processor 170 may perform control such that the left side and the right side of the shoelaces ALSa and ASLb are tightened differently in order to correct the gait or the like.

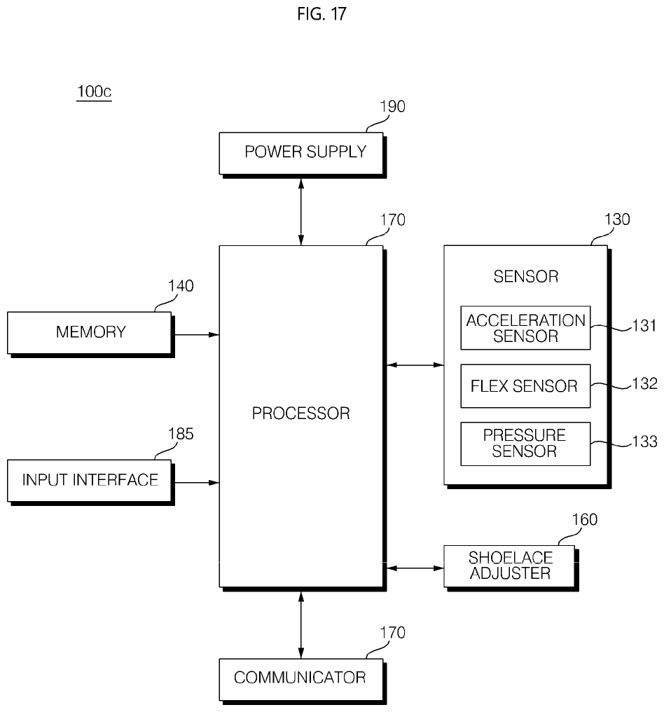

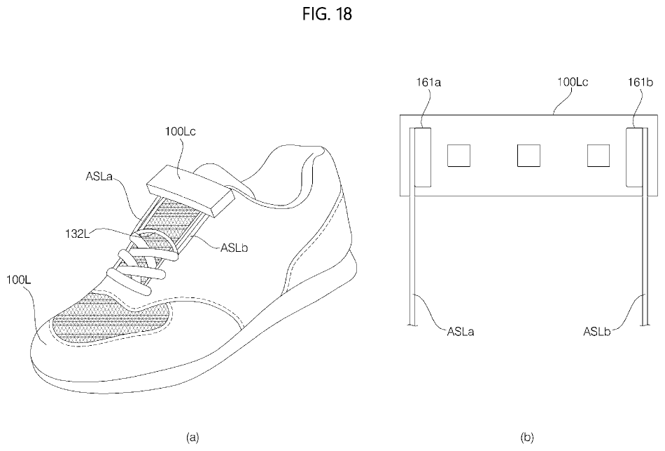

FIG. 17 is a schematic internal block diagram of still another example of the shoelace adjusting device in FIG. 2, and FIG. 18 is a view referenced to describe the method of operating the shoelace adjusting device in FIG. 17.

Referring to FIG. 17, the shoelace adjusting device 100c in FIG. 17 is similar to the shoelace adjusting device 100 in FIG. 3, but is different in that a shoelace adjuster 160, which adjusts the shoelace tightening level using electrostimulation, is provided in place of the motor 155.

Specifically, as shown in FIG. 18, the shoelace adjusting device 100c includes shoelace adjusters 161a and 161b, which are in contact with the part portions of the shoelaces ALSla and ASLb, respectively.

In one example, when a positive-polarity electric signal having the level a is applied to the shoelace adjusters 161a and 161b, the shoelaces may be tightened to the first level.

In another example, when a positive-polarity electric signal having the level b, which is lower than the level a, is applied to the shoelace adjusters 161a and 161b, the shoelaces may be tightened to the second level.

In another example, when a negative-polarity electric signal is applied to the shoelace adjusters 161a and 161b, the shoelaces may be tightened to the third level.

That is, the shoelace adjusting device 100c may determine whether the shoes 50 are being used in a running state, a walking state, or a sitting state based on sensing information from the motion sensor 131, and may control a change in the level for tightening the shoelaces ASL based on the respective states. Accordingly, the tightness of the shoelaces ASL may be automatically adjusted based on the state of the user wearing the shoes 50.

The shoelace adjusting device and the shoe including the same based on the embodiment of the present disclosure is not limited to the configurations and methods of the above-described embodiments, and all or some of the embodiments may be selectively combined to obtain various modifications.

In addition, it will be apparent that, although the preferred embodiments have been shown and described above, the present disclosure is not limited to the above-described specific embodiments, and various modifications and variations can be made by those skilled in the art without departing from the gist of the appended claims. Thus, it is intended that the modifications and variations should not be understood independently of the technical spirit or prospect of the present disclosure.

1. A shoelace adjusting device configured to adjust a shoelace attached to a shoe, the shoelace adjusting device comprising:
a motor configured to operate to adjust at least one part of the shoelace;
a sensor portion comprising a motion sensor; and
a processor configured to determine whether the shoe is being used in a running state, a walking state, or a sitting state based on sensing information from the motion sensor and to change a level for tightening the shoelace based on respective states.

2. The shoelace adjusting device of claim 1, wherein the sensor portion further comprises:
a flex sensor configured to detect a bent state of the shoelace, and
wherein the processor is configured to change a level for tightening the shoelace based on flex-sensing information from the flex sensor and state information of the shoe.

3. The shoelace adjusting device of claim 1, wherein the sensor portion further comprises:
a pressure sensor configured to detect a pressure, and
wherein the processor determines whether the shoe is being used in a running state, a walking state, or a sitting state based on sensing information from the motion sensor and pressure sensing information from the pressure sensor, and is configured to change a level for tightening the shoelace based on respective states.

4. The shoelace adjusting device of claim 3, wherein, when the pressure sensing information is equal to or greater than a first reference value for a predetermined period of time, the processor determines that a foot of a wearer of the shoe is in a swollen state, and is configured to change a level for tightening the shoelace in response to the swollen state.

5. The shoelace adjusting device of claim 1, wherein the processor further determines whether the shoe is taken off or put on based on sensing information from the motion sensor, and is configured to change a level for tightening the shoelace based on the state of taking off the shoe or the state of putting on the shoe.

6. The shoelace adjusting device of claim 1, further comprising:
a communicator configured to exchange data with a mobile terminal,
wherein the processor compares state information of a wearer of the shoe, received from the mobile terminal, with state information, determined based on sensing information from the motion sensor, calculates final state information, and is configured to change a level for tightening the shoelace based on the final state information.

7. The shoelace adjusting device of claim 6, wherein the processor receives temperature information from the mobile terminal, and is configured to change a level for tightening the shoelace based on the received temperature information.

8. The shoelace adjusting device of claim 6, wherein the processor receives humidity information from the mobile terminal, and is configured to change a level for tightening the shoelace based on the received humidity information.

9. The shoelace adjusting device of claim 1, wherein the motor comprises:
a first motor configured to operate to move one part of the shoelace; and
a second motor configured to operate to move another part of the shoelace, and
wherein the processor performs control to differently tighten a left side and a right side of the shoelace based on sensing information from the motion sensor.

10. The shoelace adjusting device of claim 1, wherein the processor is configured to change a level for tightening the shoelace based on a moving speed of the shoe.

11. The shoelace adjusting device of claim 1, wherein the processor determines whether the shoelace is a shoelace of a left shoe or a shoelace of a right shoe based on sensing information from the motion sensor, and is configured to change a level for tightening the shoelace based on determination information.

12. The shoelace adjusting device of claim 1, further comprising:
a communicator configured to exchange data with a mobile terminal,
wherein the processor receives shoelace tightness information from the mobile terminal and is configured to change a level for tightening the shoelace based on the received shoelace tightness information.

13. A shoelace adjusting device configured to adjust a shoelace attached to a shoe, the shoelace adjusting device comprising:
a first motor configured to operate to move at least one part of the shoelace;
a second motor configured to rotate in a same direction as the first motor and to operate to move the at least one part of the shoelace;
a sensor portion comprising a motion sensor; and
a processor configured to determine whether the shoe is being used in a running state, a walking state, or a sitting state based on sensing information from the motion sensor and to change a level for tightening the shoelace based on respective states.

14. The shoelace adjusting device of claim 13, wherein the sensor portion further comprises:
a flex sensor configured to detect a bent state of the shoelace, and
wherein the processor is configured to change a level for tightening the shoelace based on flex-sensing information from the flex sensor and state information of the shoe.

15. The shoelace adjusting device of claim 13, wherein the sensor portion further comprises:
a pressure sensor configured to detect a pressure, and
wherein the processor determines whether the shoe is being used in a running state, a walking state, or a sitting state based on sensing information from the motion sensor and pressure sensing information from the pressure sensor, and is configured to change a level for tightening the shoelace based on respective states.

16. The shoelace adjusting device of claim 13, wherein the processor further determines whether the shoe is taken off or put on based on sensing information from the motion sensor, and is configured to change a level for tightening the shoelace based on the state of taking off the shoe or the state of putting on the shoe.

17. The shoelace adjusting device of claim 13, further comprising:
a communicator configured to exchange data with a mobile terminal,
wherein the processor compares state information of a wearer of the shoe, received from the mobile terminal, with state information, determined based on sensing information from the motion sensor, calculates final state information, and is configured to change a level for tightening the shoelace based on the final state information.

18. A shoelace adjusting device configured to adjust a shoelace attached to a shoe, the shoelace adjusting device comprising:
a shoelace adjuster configured to operate to adjust at least one part of the shoelace;
a sensor portion comprising a motion sensor; and
a processor configured to determine whether the shoe is being used in a running state, a walking state, or a sitting state based on sensing information from the motion sensor and to change a level for tightening the shoelace based on respective states.

19. A shoe comprising the shoelace adjusting device configured to adjust a shoelace attached to a shoe,
wherein the shoelace adjusting device comprising:
a motor configured to operate to adjust at least one part of the shoelace;
a sensor portion comprising a motion sensor; and
a processor configured to determine whether the shoe is being used in a running state, a walking state, or a sitting state based on sensing information from the motion sensor and to change a level for tightening the shoelace based on respective states.
